(19) United States
(12) Patent Application Publication
(10) Pub. No.: US 2025/0383292 A1
(43) Pub. Date: Dec. 18, 2025

(54) METHOD OF IDENTIFYING ONE OR MORE BACTERIA

(71) Applicants: Agency for Science, Technology and Research, Singapore (SG); National University Hospital (Singapore) Pte Ltd, Singapore (SG)

(72) Inventors: Malini Olivo, Singapore (SG); Mahesh Arjandas Choolani, Singapore (SG); Yusong Wang, Singapore (SG); Jayakumar Perumal, Singapore (SG); Kapil Dev, Singapore (SG); Ghayathri Balasundaram, Singapore (SG); Mohesh Moothanchery, Singapore (SG); Dinish Unnimadhava Kurup Soudamini Amma, Singapore (SG)

(21) Appl. No.: 18/880,890

(22) PCT Filed: Jul. 5, 2023
(86) PCT No.: PCT/SG2023/050472

§ 371 Date: Jan. 3, 2025

(30) Foreign Application Priority Data
Jul. 5, 2022 (SG) 10202250373D

(51) Int. Cl. G01N 21/65; G01N 33/543; G01N 33/569; G16B 40/10; G16B 40/20; G16B 45/00

(52) U.S. Cl. CPC G01N 21/658; G01N 33/54346; G01N 33/56911; G16B 40/10; G16B 40/20; G16B 45/00; G01N 2333/21; G01N 2333/305

(57) ABSTRACT

A method of identifying one or more bacteria from a sample, where the one or more bacteria is suspected to be present in the sample. An aqueous suspension including a bacteria-SERS nanoparticle complex is provided, where the bacteria-SERS nanoparticle complex includes (i) a SERS nanoparticle and (ii) the one or more bacteria suspected to be present in the sample. Furthermore, the aqueous suspension is deposited on a substrate having structures coated with a SERS-active material to dispose the bacteria-SERS nanoparticle complex on the substrate. Additionally, the bacteria-SERS nanoparticle complex disposed on the substrate is irradiated to generate one or more SERS signals. Furthermore, a SERS-spectroscopic module is operable to render one or more SERS spectral data corresponding to the one or more SERS signals. Additionally, a process module is operable to identify the presence or absence of the one or more bacteria from the one or more SERS spectral data.

### CROSS-REFERENCE TO RELATED APPLICATION

This application claims the benefit of priority of Singapore patent application number 10202250373D filed 5 Jul. 2022, the contents of which being hereby incorporated by reference in its entirety for all purposes.

### TECHNICAL FIELD

Various embodiments disclosed herein relate to a method of identifying bacteria.

### BACKGROUND

Maternal vaginal-rectal colonization of Group B streptococcus (GBS) is the primary risk factor for early-onset GBS (EOGBS) disease in newborns, which may result in long-term disabilities and potentially death.

Presently, culture-based prenatal screening is being used to screen for the bacteria. However, it excludes women who have preterm delivery and correlates poorly with actual intrapartum colonization. Furthermore, as with traditional bacterial culture screening methods, it is hampered by turn-around time, low sensitivity and specificity. False positives may lead to improper usage of antibiotics, while false negatives may result in EOGBS cases. There is no universal GBS screening method in pregnancy or for point-of-care test.

Although newer techniques such as PCR methods, whole genome sequencing and MADLI-TOD MS, have been developed, large scale adoption of these techniques are hindered by investment, cost, and expertise.

In light of the above, there exists a need for an improved method of identifying bacteria that addresses or at least alleviates one or more of the above-mentioned problems.

### SUMMARY

In embodiments disclosed herein, there is provided a method of identifying one or more bacteria from a sample, wherein the one or more bacteria is suspected to be present in the sample. The method may comprise providing an aqueous suspension comprising a bacteria-SERS nanoparticle complex, wherein the bacteria-SERS nanoparticle complex comprises (i) a SERS nanoparticle and (ii) the one or more bacteria suspected to be present in the sample; depositing the aqueous suspension comprising the bacteria-SERS nanoparticle complex on a substrate having structures coated with a SERS-active material to dispose the bacteria-SERS nanoparticle complex on the substrate; irradiating the bacteria-SERS nanoparticle complex disposed on the substrate with laser to generate one or more SERS signals; having a SERS-spectroscopic module operable to render one or more SERS spectral data corresponding to the one or more SERS signals; and having a process module operable to identify the presence or absence of the one or more bacteria from the one or more SERS spectral data.

### BRIEF DESCRIPTION OF THE DRAWINGS

In the drawings, like reference characters generally refer to the same parts throughout the different views. The drawings are not necessarily drawn to scale, emphasis instead generally being placed upon illustrating the principles of various embodiments. In the following description, various embodiments of the invention are described with reference to the following drawings.

FIG. 1 is a schematic illustration of a method of identifying one or more bacteria from a sample according to embodiments disclosed herein. The method may be label-free, involving use of a sandwich surface-enhanced Raman spectroscopy (SERS) platform and machine learning. In the embodiment shown, a bacteria 101 may be dispersed in an aqueous solution such as water, to which SERS nanoparticles 103 may be added. The bacteria 101 and SERS nanoparticles 103 may form a bacteria-SERS nanoparticle complex 105. A plurality of bacteria-SERS nanoparticle complexes 105 may be formed. The bacteria-SERS nanoparticle complexes 105 may be deposited on a substrate having structures coated with a SERS-active material 107. An example of the substrate may be silicon and such structures may be silver-coated silicon nanopillar (SNP). The bacteria-SERS nanoparticle complexes 105 and the substrate having structures coated with a SERS-active material 107 may form a sandwich SERS platform, whereby at least a portion of the bacteria 101 may be positioned between the SERS nanoparticles 103 and the substrate having structures coated with a SERS-active material 107. Laser 109 may be irradiated onto the bacteria-SERS nanoparticle complexes 105 deposited on the substrate having structures coated with a SERS-active material 107 to generate one or more SERS signals 111. The one or more SERS signals 111 may be processed by a SERS-spectroscopic module operable to render one or more SERS spectral data 113 corresponding to the one or more SERS signals. The one or more SERS spectral data may in turn be processed by a process module operable to identify the presence or absence of the bacteria 101 from the one or more SERS spectral data, to provide a processed result 115. The processing may be carried out using machine learning-aided data analytics. Using a method disclosed herein, different bacterial types, which may be present in a sample, may be identified and classified clearly based on corresponding spectral signature of the bacteria.

FIG. 2 is a SERS spectra depicting detection of Pseudomonas putida (PP) bacteria on different supports of silver-coated silicon nanopillar (207), SERS nanoparticle (203), and sandwich SERS platform (205) whereby the bacteria is positioned between SERS nanoparticles and a silicon nanopillar. The spectra in the range of about 780 cm-1 to 1400 cm⁻¹ were truncated for comparison clarity. As can be seen, the sandwich SERS platform (205) provided an enhanced SERS response signal which is 4 to 5 times greater than that derived from silver-coated silicon nanopillar (207) only, and 2 to 3 times greater than that derived from SERS nanoparticle (203) only.

FIG. 3A is a scanning electron microscopy (SEM) image (top view) of a substrate comprising silver-coated silicon nanopillar (SNP), which is a component of a sandwich SERS detection platform according to embodiments disclosed herein. Scale bar of the SEM image denotes 1 μm. Insert shows an optical image of the substrate.

FIG. 3B is a scanning electron microscopy (SEM) image (side view) of a substrate comprising silver-coated silicon nanopillar (SNP), which is a component of a sandwich SERS detection platform according to embodiments disclosed herein. Scale bar of the SEM image denotes 1 μm. Insert shows an optical image of the substrate, with scale bar denoting 250 nm.

FIG. 3C is a scanning electron microscopy (SEM) image of hybrid metal nanoparticles comprising gold and silver as staining agent, which is a component of a sandwich SERS detection platform according to embodiments disclosed herein. Scale bar of the SEM image denotes 100 nm. Insert shows an optical image of the hybrid metal nanoparticles.

FIG. 3D is a UV-Vis spectrum of the hybrid metal nanoparticles shown in FIG. 3C.

FIG. 3E is a dynamic light scattering (DLS) size distribution curve of the hybrid metal nanoparticles shown in FIG. 3C. Based on DLS characterization, the hybrid metal nanoparticles had an average size of about 59 nm.

FIG. 4 shows SERS comparison of different mixing ratio of Pseudomonas putida (PP) bacteria/SERS-nanoparticle suspensions, where the spectra in the range of about 780 cm-1 to 1400 cm⁻¹ are truncated for comparison clarity. Insets are expanded views of bacterial characteristic Raman peaks at both (i) 740 cm⁻¹, and (ii) 1460 cm⁻¹ under different mixing ratio by volume (bacteria/nanoparticle) of 0 (402), 2 (404), 1 (406), 0.5 (408), 0.25 (410), 0.2 (412), and 0.1 (414). For (i), labels on vertical axis of Counts read 0, 1000, 2000, 3000, and 4000 while horizontal axis of Raman shift/cm⁻¹ reads 700, 710, 720, 730, 740, 750, 760, and 770. For (ii), labels on vertical axis of Counts read 0, 1000, 2000, 3000 and 4000 while horizontal axis of Raman shift/cm⁻¹ reads 1430, 1440, 1450, 1460, 1470, and 1480.

FIG. 5 shows sandwich SERS mapping (5× objective lens) of GBS bacteria (BAA-1138, A909), with optical density (OD)=1.2, 0.6, 0.3, and 0.15. Scale bar denotes 200 μm. The display mapping area shows part of total mapping region (about 500 μm×250 μm) after removing those areas outside of dried samples, for the purpose of highlighting regions that showed the presence of GBS bacteria.

FIG. 6A shows a first sandwich test (202103) using MRSA bacterial sample, with (i) the mapping image at 730 to 760 cm⁻¹ and (ii) a representative spectrum. Scale bar denotes 200 μm.

FIG. 6B shows a second sandwich test (202104) using MRSA bacterial sample, with (i) the mapping image at 730 to 760 cm⁻¹ and (ii) a representative spectrum. Scale bar denotes 200 μm. Comparison of the results from the first and second sandwich tests of FIG. 6A and FIG. 6B shows sandwich SERS mapping repeatability. Note: fresh MRSA bacterial samples were prepared separately for the two tests.

FIG. 7A shows a sandwich SERS mapping at 5× objective (1% laser power) with (i) the mapping image at 730 to 760 cm⁻¹ and (ii) a representative spectrum for an example of MRSA solution with OD=0.15. Scale bar denotes 200 μm.

FIG. 7B shows a sandwich SERS mapping at 50× objective (0.25% laser power) with (i) the mapping image at 730 to 760 cm⁻¹ and (ii) a representative spectrum for an example of MRSA solution with OD=0.15. Scale bar denotes 20 μm. Comparison of the results from the SERS mappings at different objectives of FIG. 7A and FIG. 7B shows that compared with 5× objective, 50× objective system gives a higher signal/noise ratio, though with a higher intensity variation, as shown in the example of MRSA solution with OD=0.15.

FIG. 8 shows sandwich SERS mapping (50× objective) of GBS bacteria (BAA-1138), with OD=0.15, 0.075, 0.0375, and 0.01875. Scale bar denotes 20 μm. Limit of Detection (LOD) of individual GBS bacteria is close to OD=0.01875. Note the mapping area is about 110 μm×100 μm.

FIG. 9 shows principal component analysis (PCA) analysis of four individual bacteria including Group B streptococcus (GBS) bacteria (BAA-1138, and BAA-611, indicated as “B1138” and “B611” respectively), Methicillin-resistant Staphylococcus aureus (MRSA) (BAA-1717) and Pseudomonas putida (PP) (ATCC, 47054). Note that each bacterial solution has the OD=0.6. This reflects the ability of sandwich SERS platform to differentiate bacterial types.

FIG. 10A shows machine learning to unmix bacterial mixture for binary mixture GBS (BAA-611) and GBS (BAA-1138) (indicated as “B611” and “B1138” respectively). Note that each GBS bacterium has the OD=0.6.

FIG. 10B shows machine learning to unmix bacterial mixture for ternary mixture composed of GBS (BAA-611), GBS (BAA-1138) (indicated as “B611” and “B1138” respectively), and MRSA (BAA-1717, TCH1516). Note that each GBS bacterium has the OD=0.6 while MRSA act as background bacteria with OD=1.2.

FIG. 10C shows machine learning to unmix bacterial mixture for quaternary mixture composed of GBS (BAA-611), GBS (BAA-1138) (indicated as “B611” and “B1138” respectively), MRSA (BAA-1717), and PP. Note that each GBS bacterium has the OD=0.6 while MRSA and PP act as background bacteria with OD=1.2 for each.

### DESCRIPTION

The following detailed description refers to the accompanying drawings that show, by way of illustration, specific details and embodiments in which the invention may be practised. These embodiments are described in sufficient detail to enable those skilled in the art to practise the invention. Other embodiments may be utilized and structural, logical, and electrical changes may be made without departing from the scope of the invention. The various embodiments are not necessarily mutually exclusive, as some embodiments can be combined with one or more other embodiments to form new embodiments.

Methods disclosed herein are based on surface-enhanced Raman spectroscopy (SERS) technique, which brings bacterial fingerprint spectral features in favor of specific recognition of bacteria. SERS could boost the detection signal with several magnitudes of enhancement for higher detection sensitivity. Together with multivariate analysis, methods disclosed herein may enable rapid detection and identification of one or more bacteria in a label-free manner.

Particularly, methods disclosed herein may allow rapid intrapartum detection of Group B Streptococcus (GBS) status in women. Through the use of rapid surface-enhanced Raman spectroscopy (SERS) mapping with machine learning-assisted data analytics, rapid, cost-effective and point-of-care diagnostics for women's health at the bedside may be achieved.

With the above in mind, various embodiments refer to a method of identifying one or more bacteria from a sample, wherein the one or more bacteria is suspected to be present in the sample.

The term “identifying” as used herein refers to a method of verifying the presence of a given molecule.

The terms “one or more” or “at least one” as used interchangeably herein refers to 1, 2, 3 or more, for example at least 1, 2, 3, 4, 5, 6, 7, 8, 9, 10, 12, 14, 15, 20, 25 or a plurality. In this connection, the term “plurality” means more than two.

The term “bacteria” as used herein includes reference to a single bacterium. The one or more bacteria may comprise a bacterium from a streptococcaceae family, pseudomonadaceae family, and/or a staphylococcaceae family. In various embodiments, the method may comprise intrapartum identification of Streptococcus agalactiae from a sample.

The term “sample”, as used herein, refers to an aliquot of material, frequently biological matrices, an aqueous solution or an aqueous suspension containing or derived from biological material. Non-limiting examples of samples may include human and animal body fluids such as whole blood, serum, plasma, cerebrospinal fluid, sputum, bronchial washing, bronchial aspirates, urine, semen, lymph fluids and various external secretions of the respiratory, intestinal and genitourinary tracts, tears, saliva, milk, white blood cells, myelomas and the like; biological fluids such as cell culture supernatants; tissue specimens which may or may not be fixed; and cell specimens which may or may not be fixed.

The method may include providing an aqueous suspension comprising a bacteria-SERS nanoparticle complex, wherein the bacteria-SERS nanoparticle complex comprises (i) a SERS nanoparticle and (ii) the one or more bacteria suspected to be present in the sample.

The SERS nanoparticle used to form the bacteria-SERS nanoparticle complex may be referred to as “SERS staining nanoparticle”, or for brevity “staining particle” and “staining agent”. By the term “nanoparticle”, it refers to a particle having a characteristic length, such as diameter, in the range of up to 100 nm.

The SERS nanoparticle may comprise a SERS-active material. As used herein, the term “SERS-active” refers to materials that enhance Raman scattering of a Raman-active molecule adsorbed thereon. In various embodiments, a SERS-active material enhances Raman scattering of a Raman-active molecule adsorbed thereon by a factor of 104, 106, 1010, or more. Examples of a SERS-active material include, but are not limited to, noble metals such as silver, palladium, gold, platinum, iridium, osmium, rhodium, ruthenium; copper, aluminum, or alloys thereof.

The SERS nanoparticle may be coated with or be formed entirely of a SERS-active material. For example, the SERS nanoparticle may be formed from a non-SERS active material, such as plastic, ceramics, composites, glass or organic polymers, and coated with a SERS-active material. The SERS nanoparticle may alternatively be formed entirely from a SERS metal selected from the group consisting of a noble metal, copper, aluminum, and alloys thereof.

In various embodiments, the SERS nanoparticle comprises a SERS active material such as silver and/or gold. In some embodiments, the SERS nanoparticle comprises, or is formed from, both silver and gold. For example, the SERS nanoparticle may be a silver or gold nanoparticle. Other than silver and gold, the SERS nanoparticle may be formed of or include any other metal capable of rendering an SERS signal.

The SERS nanoparticle may be irregular or regular in shape. In some embodiments, the SERS nanoparticle is regular in shape. For example, the SERS nanoparticle may have a regular shape such as a sphere.

Size of the SERS nanoparticle may be characterized by its diameter. In the context of a plurality of SERS nanoparticles, size of the SERS nanoparticles may be characterized by their mean diameter. The term “mean diameter” refers to an average diameter of the nanoparticles, and may be calculated by dividing sum of the diameter of each nanoparticle by the total number of nanoparticles.

In various embodiments, size of the SERS nanoparticle may be in the range of about 5 nm to about 250 nm, such as about 40 nm to about 150 nm, about 60 nm to about 100 nm, about 50 nm to about 80 nm, about 60 nm to about 80 nm, about 30 nm to about 70 nm, about 30 nm to about 60 nm, about 50 nm to about 70 nm, about 50 nm to about 60 nm, or about 60 nm.

Advantageously, the SERS nanoparticle may have surface affinity to bacteria membrane but with minimal Raman background signal, and form complex with bacteria during solution incubation. The incubation time is not particularly limited and may range from few minutes to an hour. In various embodiments, the incubation time is in the range from about 5 minutes to about 30 minutes, such as about 10 minutes to about 20 minutes, about 8 minutes to about 15 minutes, or about 10 minutes. Each bacteria-SERS nanoparticle complex may be formed from a bacterium and a SERS nanoparticle, or from a bacterium and a plurality of SERS nanoparticles. It may also be possible for each bacteria-SERS nanoparticle complex to be formed from two or more bacteria, which may be the same or different, and a plurality of SERS nanoparticles. For a bacteria-SERS nanoparticle complex formed with more nanoparticles, overall SERS signal may be higher as compared to one that is formed with less nanoparticles due to stronger plasmon effect.

The SERS nanoparticle may be modified to contain one or more functional groups on the SERS nanoparticle surface, wherein the one or more functional groups may be an amine and/or a hydroxyl. This may allow better interaction (or even binding) between the SERS nanoparticle and membrane surface of a bacterium. The interaction may include, as non-limiting examples, hydrogen binding and/or electrostatic attraction.

In various embodiments, providing the aqueous mixture comprises dispersing the sample in an aqueous medium, and mixing the sample in the aqueous medium with the SERS nanoparticle. Non-limiting examples of the aqueous medium may include phosphate buffered saline (PBS) and/or water.

The sample suspected to contain the one or more bacteria may be mixed with the aqueous medium at a volume ratio in a range of more than 0 and up to 2. For example, the volume ratio may be in the range of about 0.1 to about 2, about 0.2 to about 2, about 0.25 to about 2, about 0.5 to about 2, about 1 to about 2, about 0.1 to about 1, about 0.1 to about 0.5, about 0.1 to about 0.25, about 0.1 to about 0.2, about 0.2 to about 1, about 0.25 to about 1, or about 0.5. In specific embodiments, the sample suspected to contain the one or more bacteria may be mixed with the aqueous medium at a volume ratio of 0.5.

Methods disclosed herein may include depositing the aqueous suspension comprising the bacteria-SERS nanoparticle complex on a substrate having structures coated with a SERS-active material to dispose the bacteria-SERS nanoparticle complex on the substrate.

In various embodiments, the structures are nanostructures having at least one dimension that is in the nanometer range. At least one dimension of the nanostructures may be less than 100 nm. Examples of a nanostructure include, but are not limited to, nanopillars, nanotubes, nanoflowers, nanowires, nanofibers, nanoflakes, nanodiscs, and combinations of the aforementioned.

In some embodiments, the structures comprise or consist of nanopillars. Each of the nanopillars may have a diameter in the range of about 10 nm to about 100 nm. For example, each nanopillar may have a diameter in the range of about 20 nm to about 100 nm, about 40 nm to about 100 nm, about 50 nm to about 100 nm, about 60 nm to about 100 nm, about 70 nm to about 100 nm, or about 80 nm to about 100 nm. In some embodiments, a plurality of nanopillars are present, and the nanopillars may have an average diameter in the range from about 70 nm to about 100 nm.

Each of the nanopillars may have a height in the range of about 100 nm to about 1 μm. For example, each nanopillar may have a height in the range of about 200 nm to about 1 μm, about 300 nm to about 1 μm, about 400 nm to about 1 μm, about 100 nm to about 900 nm, about 100 nm to about 800 nm, about 100 nm to about 700 nm, about 100 nm to about 600 nm, about 100 nm to about 500 nm, or about 200 nm to about 500 nm. In some embodiments, a plurality of nanopillars are present, and the nanopillars may have an average height in the range from about 200 nm to about 500 nm.

By the term “coated with”, this means that a surface of the structures has a layer of a SERS-active material formed thereon. For example, the structures may be formed from a non-SERS active material, such as plastic, ceramics, composites, glass or organic polymers, which has a layer of SERS-active material coated thereon to render its plasmonic characteristic. It is also possible for the structures to be formed entirely from a SERS-active material. Examples of suitable SERS-active materials have already been mentioned above. Similar functional groups such as amine and/or a hydroxyl as that mentioned above may also be present on the SERS-active material surface to allow better interaction (or even binding) between the SERS-active material surface and the membrane surface of a bacterium. The SERS-active material may be the same as or different from the SERS-active material comprised in the SERS nanoparticle, which may include silver, gold and/or any other metal capable of rendering a SERS signal. In various embodiments, the SERS-active material comprises silver and/or gold. In some embodiments, the SERS-active material comprises, or is formed from silver.

In various embodiments, depositing the aqueous suspension on a substrate is carried out such that the one or more bacteria is positioned between the SERS nanoparticle and the structures. At least a portion of each bacterium may be positioned between the SERS nanoparticle and the structures. Both the SER nanoparticle and the substrate having structures coated with a SERS-active material may have affinity for the bacteria, so that in depositing the bacteria-SERS nanoparticle complex on the substrate, this may result in the bacteria being positioned between the SERS nanoparticle and the structures, so as to achieve a sandwich configuration.

In embodiments disclosed herein, the sandwich configuration may include a silicon substrate comprising silver-coated silicon nanopillars, one or more bacterial analytes, and SERS nanoparticles, whereby the one or more bacterial analytes are positioned between the SERS nanoparticles and the silver-coated silicon nanopillars of the substrate.

In various embodiments, the bacteria-SERS nanoparticle complex and the structures may be absent of an antibody, a ligand and an aptamer. Advantageously, methods disclosed herein relate to label-free detection. Using methods disclosed herein, traditional complex multistep sandwich immunoassay processes are not necessary. Entities such as antibodies, ligands (3-MBPA) and aptamers are not used, which result in less background interference for label-free detection.

Methods disclosed herein may further comprise drying the substrate after the aqueous suspension comprising the bacteria-SERS nanoparticle complex is deposited. In so doing, the aqueous medium solvent may be removed, leaving behind a dried sample comprising the bacteria-SERS nanoparticle complex on the substrate. The drying may be carried out by leaving the substrate to dry at ambient conditions, or by subjecting the substrate to an air current at a temperature of less than 60° C. In some embodiments, the drying is carried out by leaving the substrate to dry under vacuum at ambient conditions such as room temperature.

Methods disclosed herein may include irradiating the bacteria-SERS nanoparticle complex disposed on the substrate with laser to generate one or more SERS signals.

In various embodiments, the laser may comprise a wavelength ranging from 600 nm to 800 nm. The laser may, for example, comprise a wavelength of 785 nm in certain non-limiting embodiments.

Multiple SERS hotspots may exist during the irradiation, such as hotspots between the SERS nanoparticles in the bacteria-SERS nanoparticle complexes, hotspots between SERS nanoparticle and structures coated with a SERS-active material on the substrate, and/or hotspots between structures coated with a SERS-active material on the substrate. A sandwich configuration, whereby the one or more bacteria is positioned between the SERS nanoparticle and/or the structures coated with a SERS-active material in these hotspots, may be obtained. Advantageously, a large field enhancement for SERS signal amplification may be achieved with the sandwich configuration in these hotspots. By providing a bacteria-SERS nanoparticle complex, with interfacing of the bacteria-SERS nanoparticle complex with structures coated with a SERS-active material on a substrate, followed by label-free sandwich SERS analysis, methods disclosed herein are advantageous for generation of the sandwich configuration and hotspots for higher SERS signaling.

Methods disclosed herein may include having a SERS-spectroscopic module operable to render one or more SERS spectral data corresponding to the one or more SERS signals.

The one or more SERS signals may be generated from an area of the substrate instead of just a specific point on the substrate. It follows that one or more SERS spectral data may be generated from an area of the substrate. This may be carried out using SERS data mapping. In various embodiments, the SERS-spectroscopic module is operable to generate SERS spectral data in the region of about 600 cm⁻¹ to about 1800 cm⁻¹ to capture any fingerprint spectra from the bacteria.

Methods disclosed herein may include having a process module operable to identify the presence or absence of the one or more bacteria from the one or more SERS spectral data.

As mentioned above, the one or more SERS signals may be generated from an area of the substrate instead of just a specific point on the substrate. The process module may advantageously identify the presence or absence of the bacteria from an area of the substrate without the need to pinpoint a specific location on the substrate. Said differently, the SERS data mapping may involve a large area covering the sample locations, which may circumvent a slower refining process to locate the samples involving use of high magnification objective and a specific sample location.

In embodiments of the present method, an example of the steps that may be involved is described as follows. Under a 5× objective lens as a non-limiting example, (1) a check may be carried out using a bright field image of sample of bacteria-SERS nanoparticle complex disposed on a substrate. The sample may be dried prior to analysis, (2) a large mapping area (such as 0.5 mm×0.25 mm or larger) covering the sample area may be selected, (3) other mapping parameter(s) (e.g. laser, detector, exposure, accumulation number, laser power) may be set up, (4) commencement of the mapping process using the process module.

As compared to mapping under high magnification (100× objective), the present mapping condition may be significantly less demanding (e.g. at least in terms of precision for the location, focus tuning, and time), and identification that is based on a large area may render it easier to identify samples inside of the selected mapping region instead of a point. Nevertheless, methods disclosed herein may not require such high magnification of 100× for bacterial detection, and is workable with 5× and 50× for the same detection. Moreover, under a 5× objective lens, the present method can detect over a larger area for a high bacterial concentration samples. Under a 50× objective lens, the method can detect over a larger area for a low bacterial concentration samples. In other words, the present method is useful and versatile for bacterial detection in a sample regardless of whether under a 5×, 50× or even a 100× objective lens is used.

In various embodiments, having the process module operable to identify the presence or absence of the one or more bacteria from the one or more SERS spectral data may comprise training the process module to identify the presence or absence of the one or more bacteria suspected to be present in the sample.

For example, training the process module may comprise feeding multiple SERS spectral data of each respective bacterium suspected to be one of the one or more bacteria in the sample to the process module. The multiple SERS spectral data may comprise 200 SERS spectra of each respective bacterium, or more than 200 SERS spectra, such as 300, 400 or 500 SERS spectra. Accuracy of the identification may be further improved with increasing number of SERS spectral data that is provided to the process module.

As mentioned above, each bacterium may have unique SERS fingerprint features. The SERS fingerprint features comprised in the SERS spectral data may be processed by a dimensional reduction algorithm such as, but not limited to, principle component analysis (PCA), for identification of the one or more bacteria.

Performing principal component analysis may include transforming the one or more SERS spectral data into a plurality of principal components for forming a two-dimensional plot. Each point on the two-dimensional plot may be calculated based on the one or more SERS spectral data rendered from the SERS-spectroscopic module. For the purpose of PCA analysis, varying wavenumber ranges, such as 600 to 1800 cm⁻¹/600 to 900 cm⁻¹/1200 to 1800 cm⁻¹, of SERS spectral data which has known representative SERS signal contribution from the bacteria of interest may be used. From these, a most preferred range may be narrowed down for further analysis. The principal component analysis may be implemented on electronic hardware, computer software, or any combination thereof.

Reference measurements may additionally be used to generate points on the two-dimensional plot, where each reference measurement may be for an attribute of a known bacteria the reference point corresponds to. In so doing, the two-dimensional plot may include a plurality of stored reference points corresponding to respective known bacteria. In other words, the two-dimensional plot may include points calculated based on the one or more SERS spectral data rendered from the SERS-spectroscopic module for the one or more bacteria to be identified, as well as points generated from known bacteria.

Numerical analysis methods, such as linear regression based machine learning methods, for example, Multivariate Curve Resolution Ordinary Least Squares (MCR-OLS) algorithm demonstrated herein, may additionally be used to further refine the SERS spectral data for identification.

In various embodiments, having the process module operable to identify the presence or absence of the one or more bacteria from the one or more SERS spectral data may comprise having the process module isolate a set of SERS spectral data which correspond to one bacterium from another set of SERS spectral data which correspond to another bacterium.

For example, the process module may identify one or more bacteria from the sample by comparing and matching the one or more SERS spectral data generated with a SERS spectra from a known bacterium. In this regard, comparison and matching of characteristic SERS spectral peaks and/or intensity may be carried out for the identification.

As another example, a dimensional reduction algorithm such as principle component analysis (PCA) as that mentioned above may be used. The process module may identify one or more bacteria from the sample by matching each point on the two-dimensional plot which has been calculated based on the one or more SERS spectral data rendered from the SERS-spectroscopic module, against reference set points generated from SERS spectral data of known bacteria.

Advantageously, due to each bacterium having its own unique SERS fingerprint features, methods disclosed herein may be used to identify one, two, three, four or more bacteria that may be present in the sample, by comparing and matching each point on the two-dimensional plot which has been calculated based on the one or more SERS spectral data rendered from the SERS-spectroscopic module corresponding to each bacterium, against reference set points generated from SERS spectral data of known bacteria. Even without use of reference set points generated from SERS spectral data of known bacteria, it may be possible to determine number of different bacteria present in the sample may by determining number of sets of points present on different positions on the two-dimensional plot, with each set of points at a respective position representing a different bacterium.

In various embodiments, the method may comprise intrapartum identification of Streptococcus agalactiae from the sample. The specific bacteria strain has unique SERS fingerprint features, which renders its suitability for differentiation via machine-learning based strategies.

In order that the invention may be readily understood and put into practical effect, particular embodiments will now be described by way of the following non-limiting examples.

### Examples

Various embodiments refer to a method for rapid intrapartum label-free SERS detection of Group B Streptococcus (GBS) in women. As disclosed herein, SERS is first applied for GBS bacterial detection with high sensitivity and specificity, and in a label-free and/or culture-free manner.

In embodiments demonstrated herein, use of a sandwich SERS platform configuration is able to achieve a further enhanced SERS response signal by 4 to 5 times greater than that achieved from a textured SERS surface (SNP) only, and by 2 to 3 times greater than that achieved from a SERS-staining particle only.

Advantageously, the label-free SERS mapping method disclosed herein may not require precisely locating sample during measurement, and is able to cover large areas of about 500 μm×250 μm in less than 30 minutes to give reproducible results. This may mean that identification of the bacteria may be carried out quickly and efficiently.

Advantageously, use of machine learning-assisted data analytics (PCA and MCR-OLS algorithm) as disclosed herein enable effective identification, classification, and unmixing of GBS bacteria (either individual or mixture, and in the presence of background bacteria) using its SERS spectral signature.

A method of identifying one or more bacteria according to embodiments disclosed herein is a fully label-free SERS detection method. Methods disclosed herein may be easily implemented in any hospitals with basic settings, for non-invasive point-of-care (POC) GBS diagnosis and prognosis monitoring.

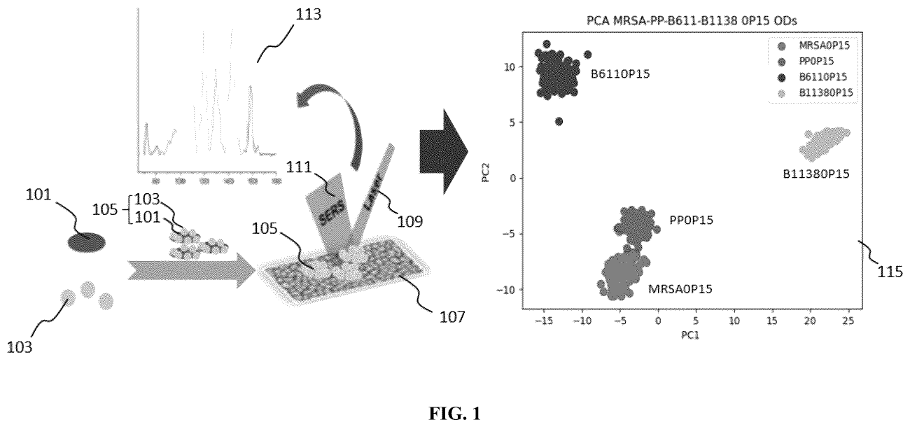

A method of identifying a bacteria, such as rapid intrapartum detection of Group B Streptococcus (GBS) status in women, according to an embodiment is shown in FIG. 1. In the embodiment shown, rapid SERS mapping with machine learning-assisted data analytics may be used for rapid, cost-effective and point-of-care diagnostics for health of women at the bedside.

Bacterial samples may be dispersed in water and mixed with SERS staining agents. The resultant mixture may be drop-cast onto SNP surface, followed by Raman laser excitation. Machine learning-aided data analytics may provide a processed result instantly, with clear identification and classification of different bacterial type based on corresponding spectral signature.

TABLE 1 provides a list of bacteria used in the experiments.

TABLE 1
List of bacteria used in embodiments disclosed herein

| | | ATCC #/ | | | Biosafety | |
|---|---|---|---|---|---|---|
| Name | Supplier | Product # | Designation | Abbreviation | Level | Remarks |
| Streptococcus | ATCC | BAA-611 | 2603 V/R | GBS | BSL-2 | — |
| Agalactiae | (USA) | | | (B611) | | |
| Streptococcus | ATCC | BAA-1138 | A909 | GBS | BSL-2 | Serotype |
| Agalactiae | (USA) | | | (B1138) | | IA; |
| | | | | | | Lancefield's |
| Pseudomonas | ATCC | 47054 | KT-2440 | PP | BSL-1 | — |
| Putida | (USA) | | | | | |
| Staphylococcus | ATCC | BAA-1717 | TCH1516; | MRSA | BSL-2 | — |
| Aureus | (USA) | | [USA300- | | | |
| subsp Aureus | | | HOU-MR] | | | |

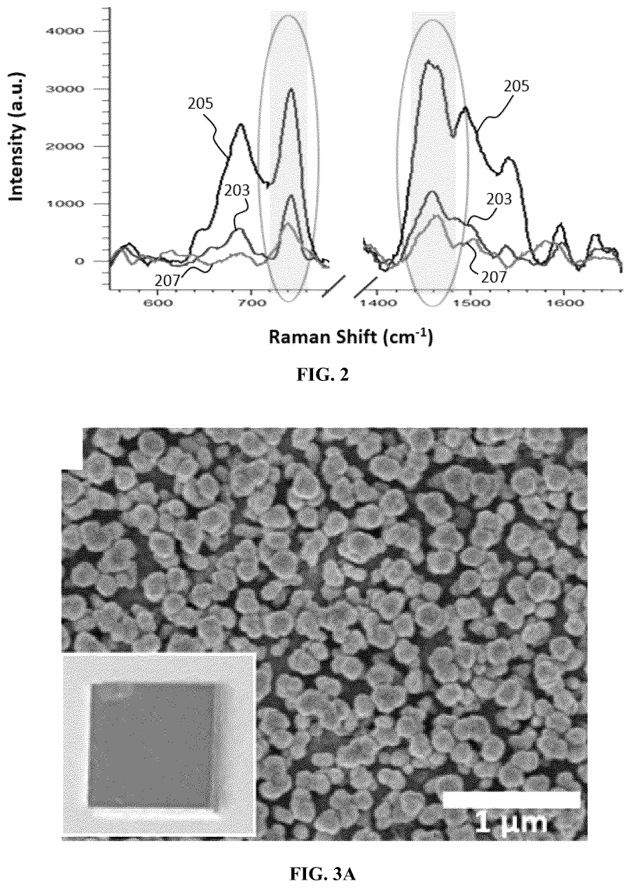

FIG. 2 shows that the sandwich configuration provided the strongest bacterial response amongst the configurations tested. Specifically, using Pseudomonas putida (P.putida, ATCC, 47054, KT-2440) as model bacterium, bacterial characteristic Raman peaks (both 740 cm-1 and 1460 cm⁻¹) under different configurations were monitored. Sandwich SERS platform (staining nanoparticles (NPs) and silver-coated silicon nanopillar (SNP)) demonstrated an enhanced SERS response signal by 4 to 5 times greater than that derived from SNP only, and 2 to 3 times greater than that derived from SERS-staining agent only.

### Example: Preparation of Components of Sandwich SERS Detection Platform

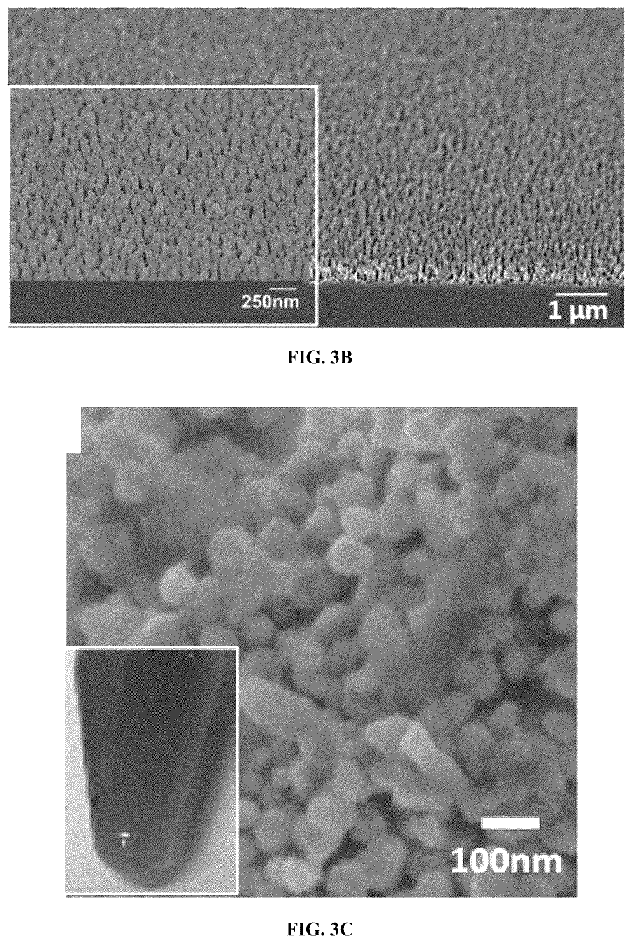

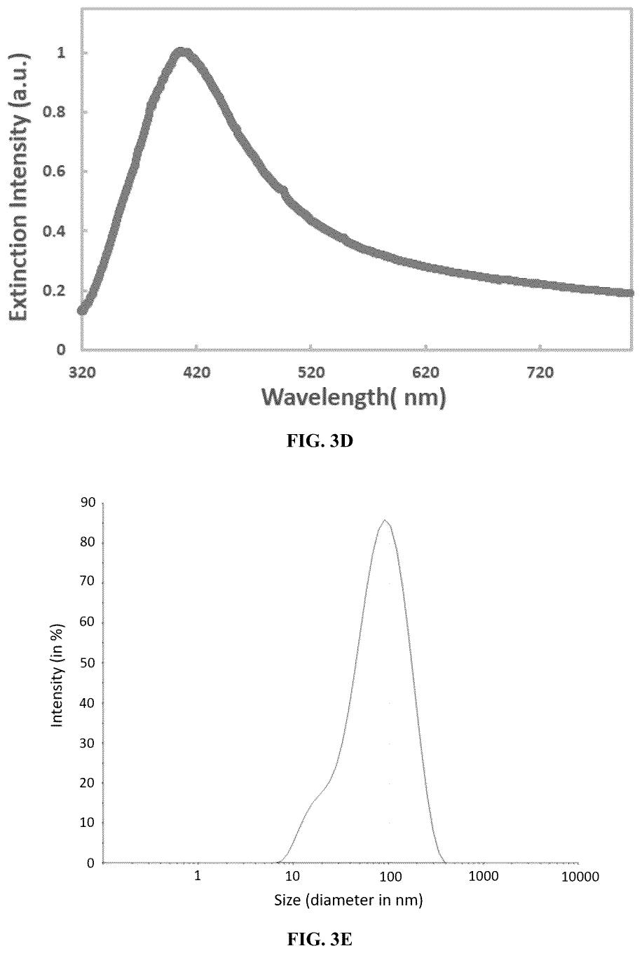

The components of sandwich SERS detection platform were in-house developed in the inventors' lab (see FIG. 3A to FIG. 3E). The components of the platform were prepared according to the following procedure.

A) Silver-coated silicon nanopillar (SNP) preparation: 1) The bare SNP substrates were fabricated at Advanced Micro Foundry (AMF) in Singapore, by blanket etch method onto silicon (Si) wafer using ICP-F etcher. Etching gas used in the fabrication process were SF₆/O₂ in a given relative ratio to etch Si uniformly to result in densely arranged Si nanopillar. The prepared Si nanopillar has a height of about 2 μm to 3 μm with a spacing of 50 nm to 150 nm among individual nanopillars. The bare SNP substrates with batch No. of S3 were used for the next processing. 2) After generating the bare SNP substrates, silver metal was coated on their surfaces, using in-house sputtering system for 300 seconds to 450 seconds with the current of 20 mA. The freshly prepared silver-coated silicon nanopillar (SNP) were consumed in 1 to 2 days. These SNP substrates were tested with 2-naphthalenethiol (2-NT) or 4-aminothiophenol (4-ATP) to know the extent of enhancement on these substrates, and the batch with the highest enhancement with weak background signal was used for the GBS study.

B) SERS staining particle preparation: SERS-staining particles were synthesized by a seed-mediated growth method. Typically, the seed solution was prepared by adding citrate solution (5 mL, 1 wt %) to boiling HAuCl₄ solution (95 mL, 0.5 mM) under vigorous stirring. After 15 minutes of boiling, the solution was cooled down to room temperature and kept at 4° C. for further processing. To prepare hybrid particles, 500 μL seed solution were added to HAuCl₄ (10 mL, 0.25 mM) solution containing HCl (10 μL, 1.0 M) at room temperature under moderate stirring. Silver nitrate (AgNO₃) (100 μL, 3 mM) and ascorbic acid (AA) (50 μL, 100 mM) were added quickly to the above solution. After this, hydrochloric amine solution (9.2 μL stock) and sodium hydroxide (30 μL, 10.0 M) were added into the above solution, and mixed well at room temperature. Silver nitrate solution (22 mM) was then quickly added into the above solution and mixed well. After the reaction solution turned a stable color, the hybrid metal nanoparticles were transferred to a 4° C. fridge for long-term storage. The hybrid metal nanoparticle as the SERS-staining nanoparticle had a size of about 59 nm in average (DLS characterization as shown in FIG. 3E).

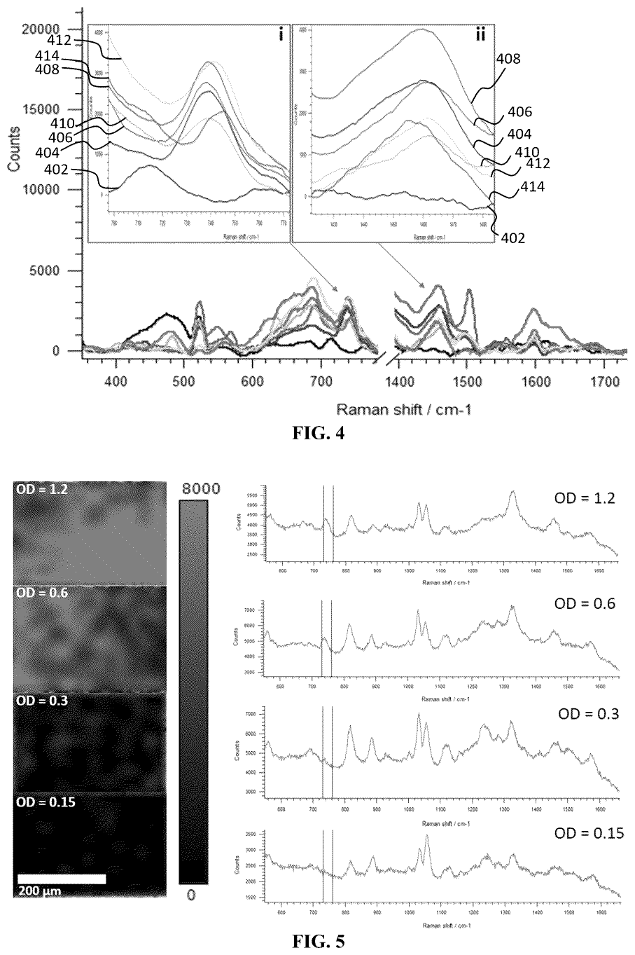

The mixing of bacterial solution and SERS-staining nanoparticles was optimized, with the following procedure. With a fixed amount of SERS-staining particle stock solution, different amount of PP bacteria (OD=0.6) solution were added and incubated in the mixture. Volume ratio of bacteria relative to SERS-staining particle solution range from 0, 0.1, 0.2, 0.25, 0.5, 1, to 2. FIG. 4 shows SERS comparison of the different mixing ratio of PP bacteria/SERS-nanoparticle suspensions, where the spectra in the range of about 780 cm-1 to 1400 cm⁻¹ are truncated for comparison clarity. The higher signal occurs for specific ratio of bacteria/SERS-staining nanoparticle solution, and the mixing ratio of 0.5 (408 in FIG. 4) was used in the later study.

### Example: SERS Mapping

Notwithstanding the above, locating the complex formed between bacteria and SERS staining agents on SNP surface for carrying out the developed sandwich SERS protocol may take up time.

To provide for a timely readout/faster screening to facilitate decision-making, a rapid and less demanding test in the form of SERS mapping was developed and optimized for practical POC diagnostic for bacterial test, with the focus on the bacteria of Group B Streptococcus Agalactiae (GBS, or S. agalactiae).

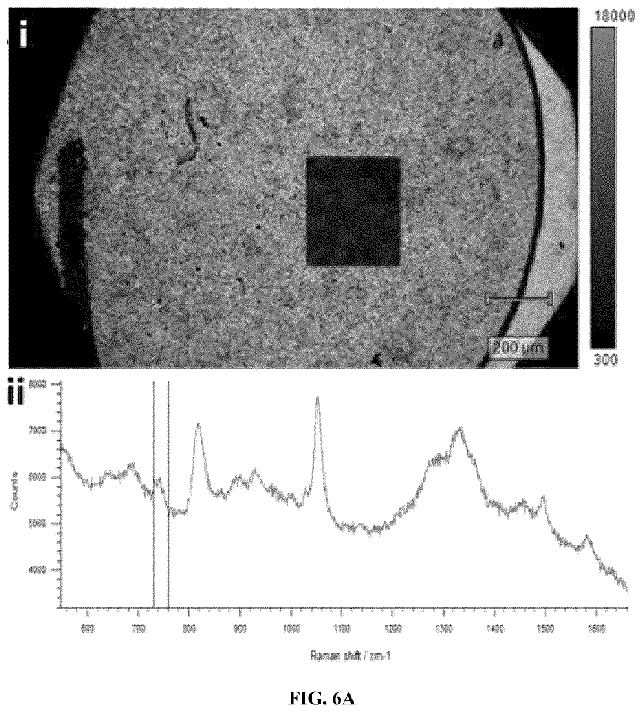

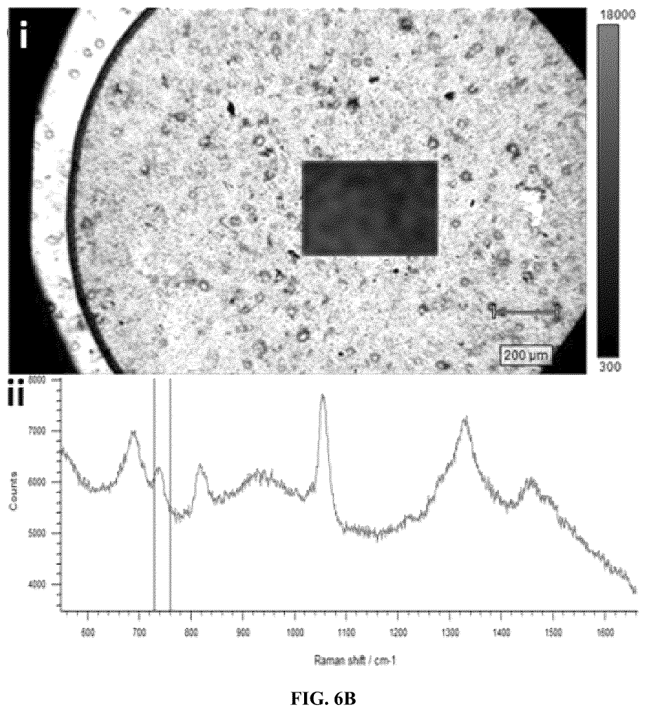

FIG. 5 shows rapid SERS mapping for the GBS bacterium (ATCC, BAA-1138, A909) in a large area (about 500 μm×250 μm), under 5× objective lens. With the concentration gradually decreasing from 1.2, 0.6, 0.3 to 0.15, SERS mapping image showed the decrease in SERS intensity. LOD of individual GBS (BAA-1138, A909) bacterium is close to OD=0.15. The label-free SERS mapping herein developed allows the method of identifying a bacteria disclosed herein to be carried out without precisely locating sample, and covers hundreds of micrometer area in less than 30 minutes. It enables the rapid readout with reproducible results (see FIG. 6A and FIG. 6B).

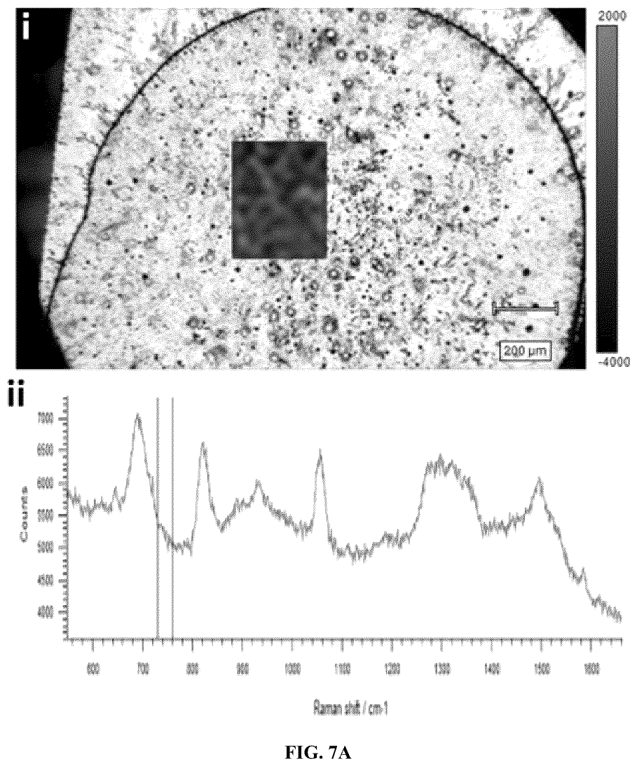

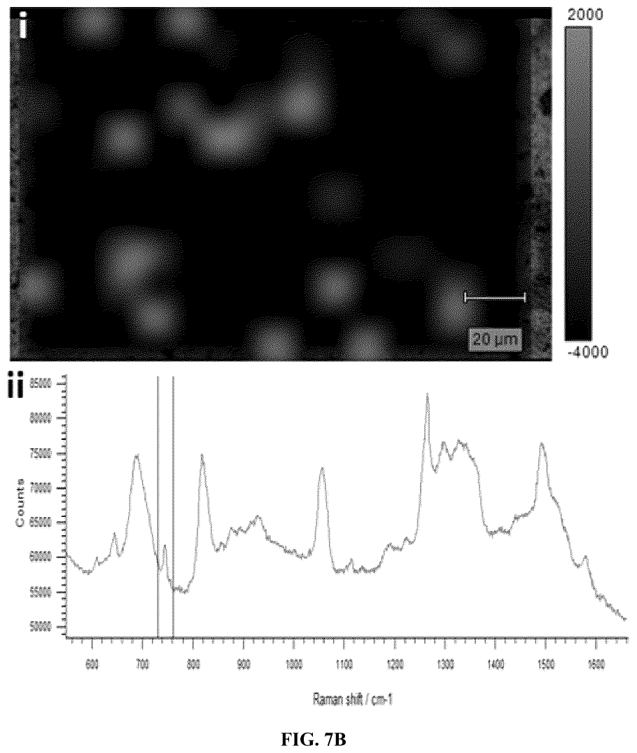

To further push the detection limit to lower concentration, high-end optical system (50× objective) was applied and the SERS mapping test was performed for the same GBS bacteria (BAA-1138, A909) (see FIG. 7A and FIG. 7B). Comparison of the results from the SERS mappings at different objectives of FIG. 7A and FIG. 7B shows that compared with 5× objective, 50× objective system gives a higher signal/noise ratio, though with a higher intensity variation, as shown in the example of MRSA solution with OD=0.15.

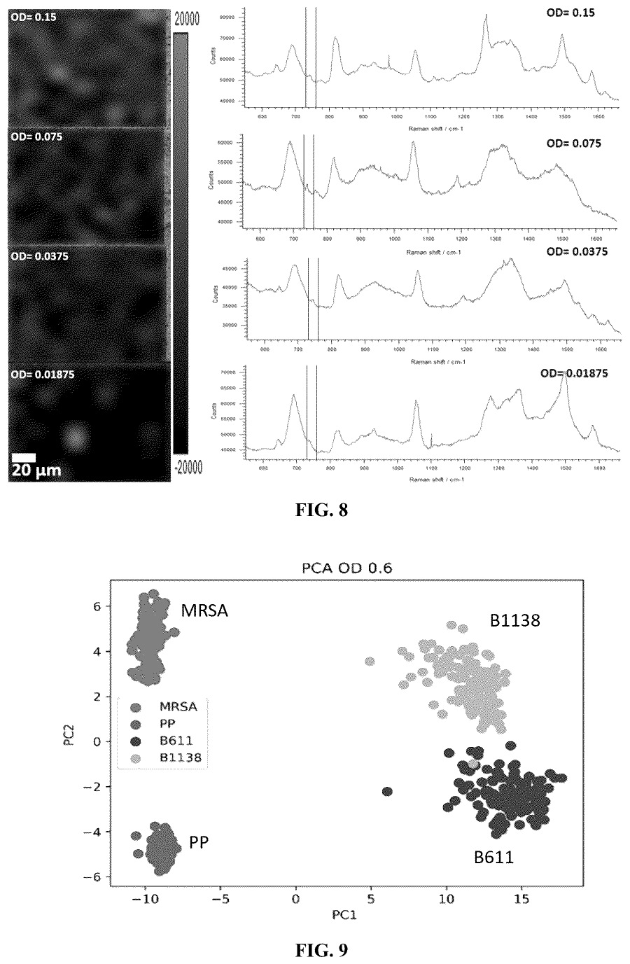

FIG. 8 shows rapid SERS mapping for an area (about 110 μm×100 μm), under 50× objective lens. With the concentration gradually decrease from 0.15, 0.075, 0.0375 to 0.01875, the SERS mapping image clearly showed the decrease in SERS intensity. LOD is close to OD=0.01875. Considering current sample preparation and OD-cfu conversion, it was close to 15×10⁵ cfu/mL, reaching the range of real clinical detection demands.

### Example: Data Analytics for GBS Bacterial Detection Using SERS Spectral Signature

Furthermore, data analytics has been developed herein for GBS bacterial detection using their respective SERS spectral signature, with about 200 spectra for each sample. Specifically, machine learning-based data processing algorithms have been developed for identification, classification, and unmixing of GBS bacteria (either individual or mixture, and in the presence of background bacteria).

In the first step, dimensional reduction algorithm, principle component analysis (PCA), has been used to identify different bacteria, including GBS (BAA-1138, A909), GBS (BAA-611, 2603 V/R), PP (47054, KT-2440), and Staphylococcus Aureus subsp Aureus (MRSA or S. aureus). FIG. 9 shows PCA analysis of these four individual bacteria based on their respective sandwich SERS spectra, with four well-separated sample clusters. This indicates that individual bacteria type (GBS, PP and MRSA) or strains (GBS (BAA-611) and GBS (BAA-1138)) may be detected early and classified.

In addition to individual bacterial analysis, bacterial mixture samples were further analyzed by “unmixing” data analysis, i.e. evaluating each bacteria constituent using linear regression based machine learning methods. Multivariate Curve Resolution Ordinary Least Squares (MCR-OLS) algorithm was used to spectrally unmix the mixture of two GBS bacteria strains (BAA-611 and BAA-1138). The data processing and spectral unmixing carried out showed that the GBS strains may be linearly unmixed effectively from each other.

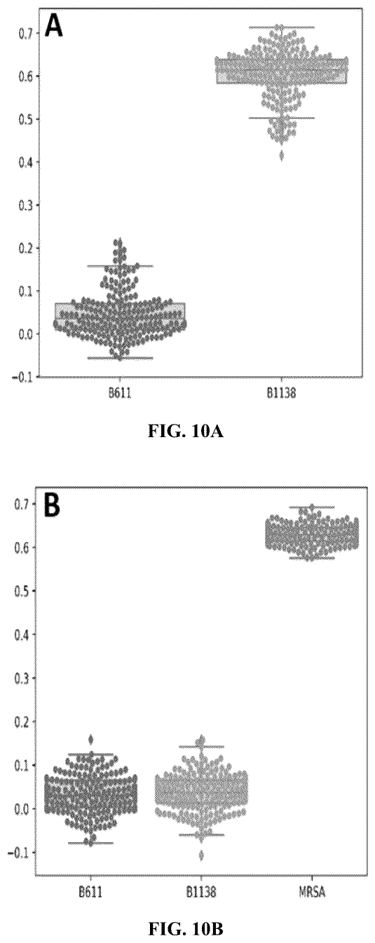

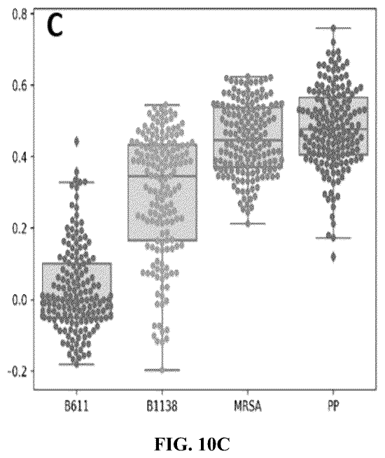

FIG. 10A shows a clear difference between the evaluated coefficients of two GBS strains (Streptococcus Agalactiae (ATCC, BAA-611, 2603 V/R) and Streptococcus Agalactiae (ATCC, BAA-1138, A909)) after applying the linear unmixing for the GBS binary mixture (each with OD=0.6). Moreover, methods disclosed herein may also identify the GBS bacteria in the presence of other bacterium MRSA.

FIG. 10B shows the differentiation of ternary bacterial mixture composed of GBS (BAA-611, 2603 V/R), GBS (BAA-1138, A909), and MRSA (BAA-1717, TCH1516), where MRSA acts as clinical bacterial background. The differentiation happened in the same way for the quaternary mixture as shown in FIG. 10C. Note that all these bacteria are BSL-2 (Biosafety Level-2), mimicking the real case of infectious bacteria.

In summary, embodiments disclosed herein provide the first SERS methodology for intrapartum label-free detection of group B streptococcus in women. Sandwich SERS mapping together machine learning-assisted data analytics may be used. Sandwich configuration may ensure an enhanced SERS signal as compared to either SNP only or SERS-staining particle only. Rapid label-free SERS mapping in 30 minutes or less according to embodiments disclosed here may allow detection of bacterial sample in the order of 10⁵ cfu/mL, meeting clinical test demands. Machine learning-assisted data analytics (PCA and MCR-OLS algorithm) may enable effective identification, classification, and unmixing of GBS bacteria (either individual or mixture, and in the presence of background bacteria) using its SERS spectral signature. The detection platform according to embodiments disclosed herein is reliable, culture-free, label-free and with high sensitivity and specificity, promising for non-invasive POC GBS diagnosis and prognosis monitoring for women's health.

By “comprising” it is meant including, but not limited to, whatever follows the word “comprising”. Thus, use of the term “comprising” indicates that the listed elements are required or mandatory, but that other elements are optional and may or may not be present.

By “consisting of” is meant including, and limited to, whatever follows the phrase “consisting of”. Thus, the phrase “consisting of” indicates that the listed elements are required or mandatory, and that no other elements may be present.

The inventions illustratively described herein may suitably be practiced in the absence of any element or elements, limitation or limitations, not specifically disclosed herein. Thus, for example, the terms “comprising”, “including”, “containing”, etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the inventions embodied therein herein disclosed may be resorted to by those skilled in the art, and that such modifications and variations are considered to be within the scope of this invention.

By “about” in relation to a given numerical value, such as for temperature and period of time, it is meant to include numerical values within 10% of the specified value.

The invention has been described broadly and generically herein. Each of the narrower species and sub-generic groupings falling within the generic disclosure also form part of the invention. This includes the generic description of the invention with a proviso or negative limitation removing any subject matter from the genus, regardless of whether or not the excised material is specifically recited herein.

Other embodiments are within the following claims and non-limiting examples. In addition, where features or aspects of the invention are described in terms of Markush groups, those skilled in the art will recognize that the invention is also thereby described in terms of any individual member or subgroup of members of the Markush group.

1. A method of identifying one or more bacteria from a sample, wherein the one or more bacteria is suspected to be present in the sample, the method comprising:
providing an aqueous suspension comprising a bacteria-SERS nanoparticle complex, wherein the bacteria-SERS nanoparticle complex comprises (i) a SERS nanoparticle and (ii) the one or more bacteria suspected to be present in the sample;
depositing the aqueous suspension comprising the bacteria-SERS nanoparticle complex on a substrate having structures coated with a SERS-active material to dispose the bacteria-SERS nanoparticle complex on the substrate;
irradiating the bacteria-SERS nanoparticle complex disposed on the substrate with laser to generate one or more SERS signals;
having a SERS-spectroscopic module operable to render one or more SERS spectral data corresponding to the one or more SERS signals; and
having a process module operable to identify the presence or absence of the one or more bacteria from the one or more SERS spectral data.

2. The method of claim 1, wherein providing the aqueous mixture comprises dispersing the sample in an aqueous medium, and mixing the sample in the aqueous medium with the SERS nanoparticle.

3. The method of claim 1, wherein the SERS nanoparticle comprises silver and/or gold.

4. The method of claim 2, wherein the sample suspected to contain the one or more bacteria is mixed with the aqueous medium at a volume ratio in a range of more than 0 and up to 2.

5. The method of claim 1, wherein the bacteria-SERS nanoparticle complex and the structures are absent of an antibody, a ligand and an aptamer.

6. The method of claim 1, wherein the structures comprise nanopillars.

7. The method of claim 1, wherein the SERS-active material comprises silver and/or gold.

8. The method of claim 1, wherein depositing the aqueous suspension on a substrate is carried out such that the one or more bacteria is positioned between the SERS nanoparticle and the structures.

9. The method of claim 1, wherein the laser comprises a wavelength ranging from 600 nm to 800 nm.

10. The method of claim 1, wherein having the process module operable to identify the presence or absence of the one or more bacteria from the one or more SERS spectral data comprises training the process module to identify the presence or absence of the one or more bacteria suspected to be present in the sample.

11. The method of claim 10, wherein training the process module comprises feeding multiple SERS spectral data of each respective bacterium suspected to be one of the one or more bacteria in the sample to the process module.

12. The method of claim 11, wherein the multiple SERS spectral data comprises 200 SERS spectra of each respective bacterium.

13. The method of claim 1, wherein having the process module operable to identify the presence or absence of the one or more bacteria from the one or more SERS spectral data comprises having the process module isolate a set of SERS spectral data which correspond to one bacterium from another set of SERS spectral data which correspond to another bacterium.

14. The method of claim 1, wherein the one or more bacteria comprise a bacterium from a streptococcaceae family, pseudomonadaceae family, and/or a staphylococcaceae family.

15. The method of claim 1, wherein the method comprises intrapartum identification of Streptococcus agalactiae from the sample.
